(19) United States
(12) Patent Application Publication
(10) Pub. No.: US 2008/0274240 A1
(43) Pub. Date: Nov. 6, 2008

(54) ADAPTIVE CONTROLLER AND EXPERT SYSTEM FOOD PROCESSING

(76) Inventors: Omar Germouni, Chicago, IL (US); James T.C. Yuan, Boothwyn, PA (US); Joseph E. Paganessi, Burr Ridge, IL (US)

Correspondence Address: AIR LIQUIDE INTELLECTUAL PROPERTY DEPT., 2700 POST OAK BLVD., SUITE 1800, HOUSTON, TX, 77056, US

(21) Appl. No.: 11/744,077

(22) Filed: May 3, 2007

(51) Int. Cl. G05B 13/02; G01N 33/02; G06N 3/02; G05B 11/32

(52) U.S. Cl. 426/231; 700/117; 700/207; 99/486; 99/487

(57) ABSTRACT

A method and apparatus for controlling efficacy of a food processing system. The method includes, responsive to receiving set points from an automatic expert system, operating an adaptive controller to perform steps including comparing the set points from the automatic expert system to process variables for the food processing system. The steps also include generating an error signal indicating a difference between the compared process variables and the set points and adjusting control settings for the food processing system using the error signal and one or more control parameters. The adaptive controller is configured to automatically modify the control parameters in response to a detected change in at least one of the set points and the process variables.

### BACKGROUND

Modern food processing typically includes complex processes which may require simultaneous control of a large number of process variables. The food processes may be designed to control preservation of food products, for example, by providing a preservative to a food product such as a liquid food product. The food processes may also be designed to prevent contamination of the food product. Contamination of the food product may be prevented, for example, by controlling the temperature of the food product during the food processing.

In some cases, food processing may be controlled manually, for example, by a technician that manually adjusts process control settings. However, manual control of food processing may be difficult to effectively and efficiently perform. For example, mechanisms which cause food contamination during food processing may not be fully understood, thereby making manual selection of appropriate process control settings difficult. Also, where a new process is implemented, the reaction of the process to various control changes under differing process conditions may not be fully understood. Furthermore, manual control may not provide adequate response time to effectively manage changes in the process conditions.

In some cases, simple controllers may be used to improve process control in a food processing environment. However, such controllers are typically simple single-input, single-output controllers which may not provide adequate response time for changes in process variables. Such controllers may also fail to compensate for interactions between different process variables.

Accordingly, what is needed is an improved method and apparatus for controlling a food processing system.

### SUMMARY

Embodiments of the invention provide a method and apparatus for controlling efficacy of a food processing system. In one embodiment, the method includes, responsive to receiving set points from an automatic expert system, operating an adaptive controller to perform steps including comparing the set points from the automatic expert system to process variables for the food processing system. The steps also include generating an error signal indicating a difference between the compared process variables and the set points and adjusting control settings for the food processing system using the error signal and one or more control parameters. The adaptive controller is configured to automatically modify the control parameters in response to a detected change in at least one of the set points and the process variables.

One embodiment of the invention also provides an adaptive controller configured to control efficacy of a food processing system. The controller includes a computer readable storage medium including a plurality of instructions and a processor, which, when executing the plurality of instructions, is configured to receive set points from an automatic expert system. The processor is further configured to compare the set points from the automatic expert system to process variables for the food processing system. Comparing includes generating an error signal indicating a difference between the process variables and the set points. The processor is also configured to adjust control settings for the food processing system using the error signal and one or more control parameters. The adaptive controller is configured to automatically modify the control parameters in response to a detected change in at least one of the set points and the process variables.

Another embodiment of the invention provides a method for generating set points used by an adaptive controller for controlling efficacy of a food processing system. The method includes receiving process variables from the food processing system and comparing the process variables from the food processing system to quality control metrics derived from the food processing system. The method further includes, based upon a comparison of the process variables and the quality control metrics, generating the set points used by the adaptive controller for controlling the food processing system. The method also includes providing the set points to the adaptive controller.

One embodiment of the invention provides an automatic expert system configured to generate set points used by an adaptive controller for controlling efficacy of a food processing system. The expert system includes a computer readable storage medium including a plurality of instructions and a processor, which, when executing the plurality of instructions, is configured to receive process variables from the food processing system. The processor is also configured to compare the process variables from the food processing system to quality control metrics derived from the food processing system and based upon a comparison of the process variables and the quality control metrics, generate the set points used by the adaptive controller for controlling the food processing system. The processor is further configured to provide the set points to the adaptive controller.

One embodiment of the invention provides a food processing system. The food processing system includes a chamber in which food processing is performed and a plurality of sensors configured to provide process variables for the food processing system. The system also includes an automatic expert system configured to receive the process variables for the food processing system and receive quality control metric for the food processing system. The expert system is further configured to compare the process variables from the food processing system to quality control metrics derived from the food processing system and, based upon a comparison of the process variables and the quality control metrics, generate the set points used by the adaptive controller for controlling the food processing system. The system further includes an adaptive controller configured to receive the set points from the automatic expert system and compare the set points to the process variables for the food processing system. Comparing includes generating an error signal indicating a difference between the process variables and the set points. The controller is further configured to adjust control settings for the food processing system using the error signal and one or more control parameters. The adaptive controller is configured to automatically modify the control parameters in response to a detected change in at least one of the set points and the process variables.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 illustrates a food processing system according to one embodiment of the invention;

FIG. 2 illustrates a control system for a food processing system according to one embodiment of the invention;

FIGS. 3A-3B illustrate aspects of an adaptive controller according to one embodiment of the invention; and

FIGS. 4A-4B illustrate aspects of an expert system according to one embodiment of the invention.

### DESCRIPTION OF PREFERRED EMBODIMENTS

Embodiments of the invention generally provide a method and apparatus for controlling efficacy of a food processing system. In one embodiment, the method includes, responsive to receiving set points from an automatic expert system, operating an adaptive controller to perform steps including comparing the set points from the automatic expert system to process variables for the food processing system. The steps also include generating an error signal indicating a difference between the compared process variables and the set points and adjusting control settings for the food processing system using the error signal and one or more control parameters. The adaptive controller is configured to automatically modify the control parameters in response to a detected change in at least one of the set points and the process variables.

In the following, reference is made to embodiments of the invention. However, it should be understood that the invention is not limited to specific described embodiments. Instead, any combination of the following features and elements, whether related to different embodiments or not, is contemplated to implement and practice the invention. Furthermore, in various embodiments the invention provides numerous advantages over the prior art. However, although embodiments of the invention may achieve advantages over other possible solutions and/or over the prior art, whether or not a particular advantage is achieved by a given embodiment is not limiting of the invention. Thus, the following aspects, features, embodiments and advantages are merely illustrative and are not considered elements or limitations of the appended claims except where explicitly recited in a claim(s). Likewise, reference to “the invention” shall not be construed as a generalization of any inventive subject matter disclosed herein and shall not be considered to be an element or limitation of the appended claims except where explicitly recited in a claim(s).

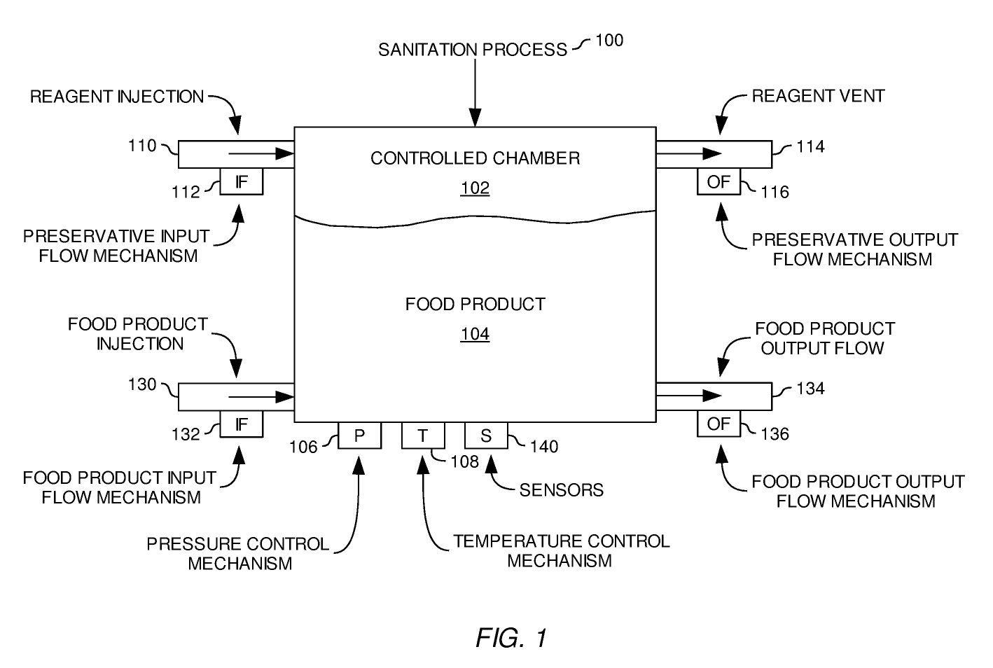

One embodiment of the invention is implemented as a program product (e.g., a distributed program product) for use with a system such as, for example, food processing system 100 shown in FIG. 1 and described below. The program(s) of the program product defines functions of the embodiments (including the methods described herein) and can be contained on a variety of computer-readable storage media. Illustrative computer-readable storage media include, but are not limited to: (i) non-writable storage media (e.g., read-only memory devices within a computer such as CD-ROM disks readable by a CD-ROM drive) on which information is permanently stored; (ii) writable storage media (e.g., floppy disks within a diskette drive or hard-disk drive) on which alterable information is stored. Such computer-readable storage media, when carrying computer-readable instructions that direct the functions of the present invention, are embodiments of the present invention. Other media include communications media through which information is conveyed to a computer, such as through a computer or telephone network, including wireless communications networks. The latter embodiment specifically includes transmitting information to/from the Internet and other networks. Such communications media, when carrying computer-readable instructions that direct the functions of the present invention, are embodiments of the present invention. Broadly, computer-readable storage media and communications media may be referred to herein as computer-readable media.

In general, the routines executed to implement the embodiments of the invention, may be part of an operating system or a specific application, component, program, module, object, or sequence of instructions. The computer program of the present invention typically is comprised of a multitude of instructions that will be translated by the native computer into a machine-readable format and hence executable instructions. Also, programs are comprised of variables and data structures that either reside locally to the program or are found in memory or on storage devices. In addition, various programs described hereinafter may be identified based upon the application for which they are implemented in a specific embodiment of the invention. However, it should be appreciated that any particular program nomenclature that follows is used merely for convenience, and thus the invention should not be limited to use solely in any specific application identified and/or implied by such nomenclature.

FIG. 1 illustrates a food processing process 100 according to one embodiment of the invention. In the depicted embodiment, the process 100 is a sanitation process, however, embodiments of the invention may be utilized with any type of process. In one embodiment, the process 100 may be performed in a controlled chamber 102 containing a food product 104. The food product 104 may be any type of food product including a solid food product, liquid food product, mixed solid and liquid food product, etc.

The chamber 102 may include inputs for preservative injection 110 and food product injection 130. The preservative may interact with the food product 104 by direct contact with the food product 104 or through a solid interface. The preservative injection 110 may be controlled by a preservative input flow mechanism 112 such as a valve or a pump. Similarly, the food product injection 130 may be controlled by a food product input flow mechanism 132 such as a valve or a pump.

The chamber 102 for the process may also include a preservative vent 114 and a food product output flow 134. The preservative vent 114 may be used to vent and/or recycle any unused preservative. The preservative vent 114 may be controlled by a preservative output flow mechanism 116 while the food product output flow 134 may be controlled by a food product output flow mechanism 136. The mechanisms 116, 136 may include any type of flow control mechanism such as a valve or pump. The pressure and temperature within the chamber 102 may be controlled by a pressure control mechanism 106 and/or by a temperature control mechanism 108. The temperature control mechanism 108 may include a heating element which may be used to heat the chamber 102 or a heating fluid which may be injected into the controlled chamber 102. Sensors 140 may be used to provide feedback about process variables (PV) such as temperature, pressure, residence time, fluid density, and solubility of the food product 104.

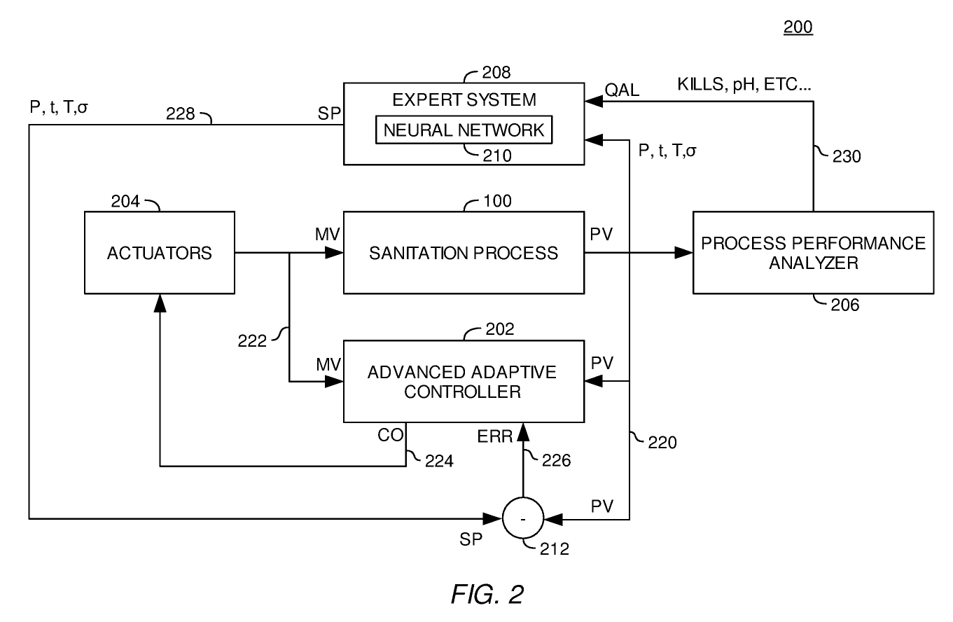

FIG. 2 illustrates a control system 200 for the process 100 according to one embodiment of the invention. As depicted, the control system 200 may include an advanced adaptive controller 202, expert system 208, process performance analyzer 206, and actuators 204 for controlling the process 100. In one embodiment, the expert system 208 is generally configured to provide set points 228 to the controller 202. To this end, one embodiment of the expert system 208 may include a neural network 210 which may be used to provide the set points 228 to the controller 202.

As described above, sensors 140 may be used to provide information about process variables (PV) 220 such as pressure (P), temperature (T), residence time (t), solubility (σ), and fluid density. The process variables 220 may be received by the controller 202, process performance analyzer 206, and expert system 208. The controller 202 may control the process 100 by providing control outputs (CO) 224 to actuators 204 and/or other control devices. The actuators 204 may, for example, control the flow of fluids into the chamber 102 via control mechanisms 112, 116, 132, 136 and may also control pressure and temperature in the chamber 102 via control mechanisms 106, 108. Outputs of the actuators 204 may be manipulated variables (MV) 222 which affect the process 100 and which may also be monitored by the controller 202 to ensure that the control outputs 224 are being properly implemented by the actuators 204.

In one embodiment, the controller 202 may compare 212 the process variables 226 to the set points 228 provided by the expert system 208. The set points 228 may indicate desired settings for the process variables 220 being measured. The comparison 212 between the process variables 220 and the set points 228 may provide an error signal 226. If the difference between a given process variable 220 and set point 228 is relatively large, the error signal 226 for that variable may also be relatively large. Similarly, if the difference between the process variable 220 and the set point 228 is relatively small, then the error signal 226 may also be relatively small. Whether an error signal is considered relatively large or small will depend on a given application, as will be appreciated by those skilled in the art.

Based on the error signal 226, the controller 202 may be configured to modify one or more of the control outputs 224 in order to reduce the error signal 226. For example, if the error signal 226 indicates that the measured pressure within the chamber 102 is below the set point 228 for the desired pressure, then the controller 202 may be configured to change the control outputs 224 in a manner that increases the pressure within the chamber 102. When modifying the control outputs 224, the controller 202 may use a gain control parameter to determine how large the change in the one or more control outputs 224 should be. For example, if the gain is relatively large, then the corresponding change in the control outputs 224 for a given error signal 226 may also be relatively large. If the gain is relatively small, then the corresponding change in the control outputs 224 for a given error signal 226 may also be relatively small. Appropriate values for the gain of the controller 202 may be selected according a given application, as will be appreciated by those skilled in the art. In general, the gain should be selected to ensure that the desired process parameter values are maintained within acceptable limits, but not so high as to induce “jitter” in the actuators 204 or adversely affect the processing of the food product (which may be sensitive to sudden and/or sufficiently large changes in process parameters). In one embodiment, the gain of the controller 202 may be between 1 and 10.

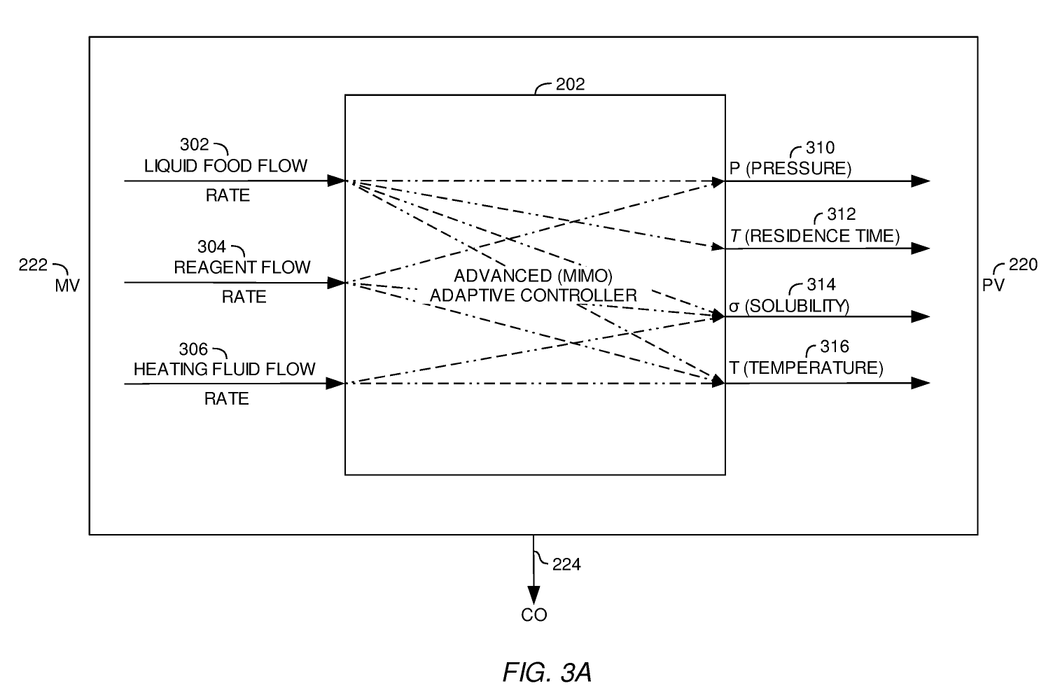

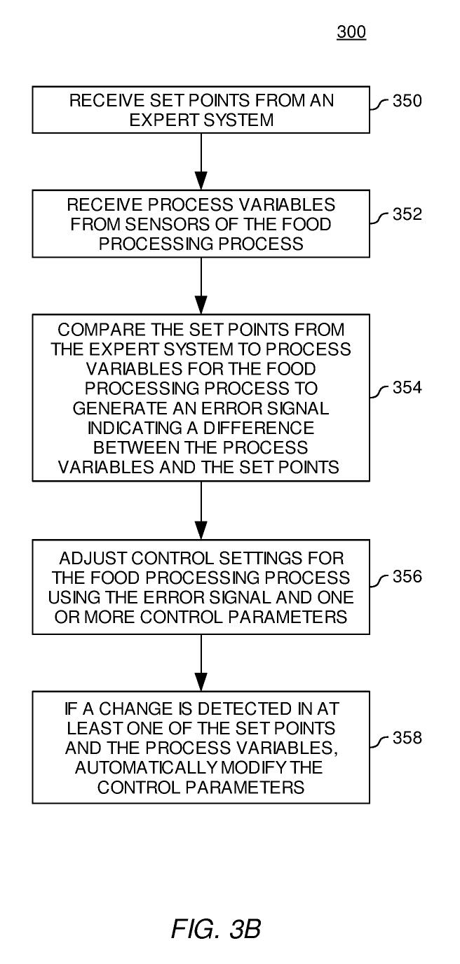

FIG. 3A is a block diagram depicting the controller 202 according to one embodiment of the invention. The controller 202 may be a multiple input, multiple output controller 202. Thus, for example, the controller 202 may receive multiple process variables 220 for pressure 310, residence time 312, solubility 314 and temperature 316 and use the multiple process variables 220 to generate multiple control outputs 224. The controller 202 may also receive multiple manipulated variables 222 (e.g., from the actuators 204 and/or other devices) such as a liquid food flow rate 302, reagent flow rate 304, and heating fluid flow rate 306. The manipulated variables 222 may also be used to determine the magnitude of a desired change in control outputs 224 for reducing the error signal 226. For example, the manipulated variables 222 may indicate whether a previous change in the control outputs 224 was correctly implemented by the actuators 204, and if not, the control outputs 224 may be further adjusted by the controller 202 to obtain the desired values of the manipulated variables 222.

FIG. 3B is a flow diagram depicting a process 300 for operating the controller 202 according to one embodiment of the invention. The process 300 may, for example, be implemented by hardware of the controller 202, software executed by the controller 202, firmware executed by the controller 202, or a combination thereof. The process 300 may begin at step 350 where set points 228 for the food processing process 100 are received from the expert system 208. At step 352, process variables 220 for the food processing process 100 may be received, e.g., from sensors 140 for the process 100.

At step 354, the set points 228 from the expert system 208 may be compared to the process variables 220 for the food processing process 100. The comparison may be used to generate the error signal 226 indicating the difference between the process variables 220 and the set points 228. For example, a given set point 228 may indicate a desired temperature for the process 100. The corresponding process variable 220 may be a temperature reading from a temperature sensor. The comparison may be performed by subtracting the desired temperature from the measured temperature to determine if the food processing process 100 is being performed at the desired temperature.

At step 356, the control outputs 224 for the food processing process 100 may be adjusted using the error signal 226 and one or more control parameters. For example, one of the control parameters may be the gain of the controller 202. The gain may indicate the magnitude of a change made to the control settings in response to the magnitude of the error signal 226. If the error signal 226 is large (e.g., if the measured temperature is much smaller than the desired temperature), then the gain may indicate that a large change should be made to the control outputs 224 to compensate (e.g., the control outputs 224 provided to the temperature control mechanism 108 may be changed to cause the mechanism 108 to increase the temperature for the sanitation process 100 quickly).

As mentioned above, in one embodiment, the controller 202 may also be adaptive. The controller 202 may adapt by automatically and autonomously modifying the control parameters (including the gain) used to set the control outputs 224 for the food processing process 100. Modifying the control parameters may also be referred to as tuning. In one embodiment, the controller 202 may be configured to automatically modify the control parameters in response to detecting a change in the set points 228 or the process variables 220 for the process 100, as depicted at step 358 of the process 300. Thus, tuning may be performed when new set points 228 are received from the expert system 208 or when conditions of the food processing process 100 change. By automatically modifying the control parameters, the controller 202 may be better able to cope with changes or nonlinearities in the behavior of the process 100. In some cases, tuning may also be performed periodically by the controller 202 (e.g., the controller 202 may tune the control parameters with a given frequency).

In some cases, constraints may be placed on changes made by the controller 202 to the control parameters and/or control outputs 224. The constraints on the changes made by the controller 202 may ensure that conditions of the process 100 are maintained within desired ranges and that no sudden, undesirable changes are made to the process 100. For example, where sudden changes are made to the process 100 (e.g., increasing the process temperature too quickly), the process reaction to the sudden changes may make the resulting food product 104 unusable, for example, by spoiling the food product 104.

In one embodiment, to prevent the controller from making large changes to the process 100, the controller gain may be limited to a range between 1 and 10 (e.g., when the controller 202 is tuned, the gain may not be changed to a value less than 1 or a value greater than 10). The controller 202 may also be configured to make changes to the control outputs 224 in steps. For example, the controller 202 may perform changes to the control outputs 224 in three steps including a first step for the lower range of the change, a second step for middle range of the change, and a third step for the upper range of the change. In some cases, the controller 202 may also be configured to make changes to the control parameters only after the process 100 has settled to a steady state. For example, after the control parameters for temperature control have been tuned, subsequent tuning may not be performed until the temperature of the process 100 stabilizes around a given level (e.g., the tuning may not be performed until the temperature of the process 100 is maintained for a predefined period of time within predefined upper and lower limits relative to the temperature set point). In one embodiment, by not performing subsequent tuning until the process 100 has settled to a steady state, an instrumented system (e.g., process performance analyzer 206) downstream of the process 100 may be able to provide quality variables which may be further used to improve control of the process 100.

In one embodiment, because the controller 202 may be a multiple input, multiple output controller, where two or more of the process variables 220 and manipulated variables 222 interact with each other, the controller 202 may compensate for the interaction accordingly. For example, in some cases, a change in temperature in the chamber 102 may cause a change in pressure in the chamber 102. Where the controller 202 detects an error signal 226 in the temperature and/or pressure of the chamber 102, the controller may modify the control outputs 224 in a manner that reduce the magnitude of the error signal 226 while compensating for the interaction between the temperature and pressure interaction within the chamber 102.

In one embodiment, the controller 202 may only use process variable inputs 220, manipulated variable outputs 222, and set points 228 provided by the expert system 208 to control the process 100 without relying on a model of the process 100. Thus, the controller 202 may be configured to control processes where no model or where only an incomplete model of a given process is available. Because the controller 202 may only use process variable inputs 220, manipulated variable outputs 222, and set points 228, the controller 202 may perform tuning and calculate control outputs 224 in real time, thereby allowing the controller 202 to quickly adapt to and compensate for any changes in the process 100. Because the controller 202 may be adaptive and operate without predefined models, the controller 202 may be effective at rejecting any disturbances in the process 100 (e.g., transitory changes in process variables 220) and compensating for any non-linearity in control of the process 202.

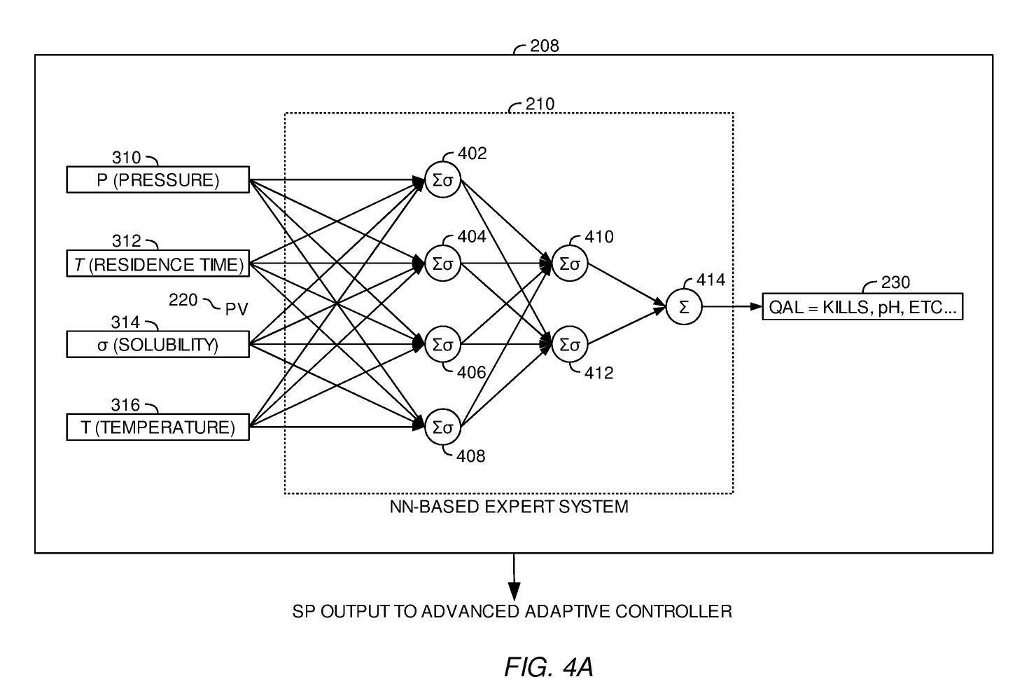

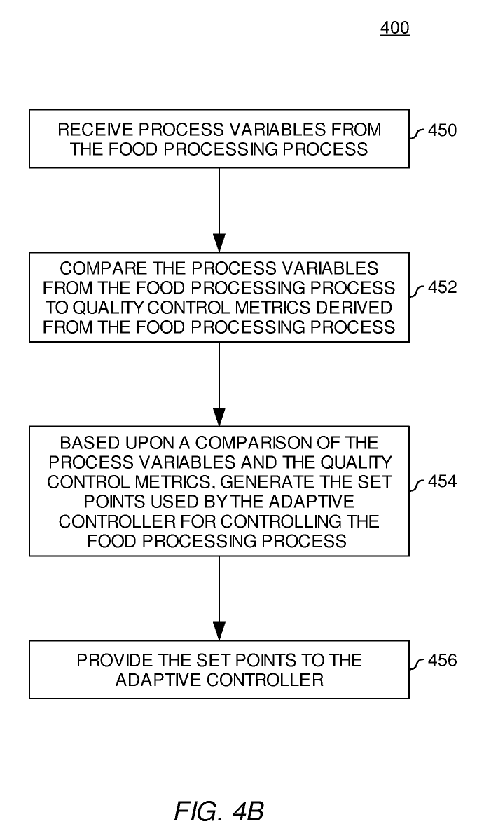

As described above, the expert system 208 may be used to provide set points 228 for the process 100 to the advanced adaptive controller 202. FIG. 4A illustrates an expert system 208 according to one embodiment of the invention. FIG. 4B illustrates a process 400 which may be used by the expert system 208 to provide the set points 228 according to one embodiment of the invention. The process 400 begins at step 450 where process variables 228 are received from the food processing process 100. At step 452, the process variables from the food processing process 100 may be compared to quality control metrics 230 derived from the food processing process 100. The quality control metrics 230 may include microbiological data such as a number of bacteria kills, the pH of the food product 104, or any other appropriate metric. In one embodiment, the quality control metrics 230 may be obtained manually, e.g., by technicians trained to sample and analyze the food product 104, thereby producing the quality control metrics 230. Optionally, the quality control metrics may be obtained automatically from the process performance analyzer 206 which may include any appropriate sensors/systems for analyzing the quality of the food processing process 100.

At step 454, based upon the comparison of the process variables 220 and the quality control metrics 230, the set points 228 used by the adaptive controller 202 for controlling the food processing process 100 may be generated and at step 456 the set points 228 may be provided to the adaptive controller 202. In one embodiment, the expert system 208 may be configured to limit the frequency with which changes are made to the set points 228 used by the controller 202. For example, the expert system 208 may make changes to the set points 228 only after the process variables 220 have reached a steady state, thereby allowing the process 100 to adjust to any previous changes in the set points 228 before subsequent changes are made. Optionally, the expert system 208 may perform set point changes more slowly than the controller's control output changes by an order of magnitude. For example, if the controller 202 changes the control outputs once a minute, then the expert system 208 may perform set point changes once every ten minutes.

In one embodiment of the invention, the expert system 208 may use a neural network 210 to perform set point calculations. As depicted in FIG. 4A, the network 210 may include a plurality of perceptrons 402-414. In one embodiment, the perceptrons 402-414 may be arranged in multiple layers including an input layer formed from perceptrons 402, 404, 406, 408, a hidden layer formed from perceptrons 410, 412, and an output layer formed from perceptron 414. A back-propagation algorithm may be used by the network 210 to calculate the set points 228. The number of layers, perceptrons, and weights used by the neural network 210 may be modified according to design considerations known to those skilled in the art.

In some cases, the expert system 208 may be configured to store and recall combinations of set points 228 that provide optimal process performance. Furthermore, in one embodiment, the expert system 208 may be enabled or disabled as desired. For example, the expert system 208 may initially be enabled to calculate appropriate set points 228. After the set points 228 have been calculated, the expert system 208 may be disabled while the adaptive controller 202 controls the process 100 using the calculated set points 228. The expert system 208 may subsequently be periodically enabled as desired in order to recalculate appropriate set points 228.

Preferred processes and apparatus for practicing the present invention have been described. It will be understood and readily apparent to the skilled artisan that many changes and modifications may be made to the above-described embodiments without departing from the spirit and the scope of the present invention. The foregoing is illustrative only and that other embodiments of the integrated processes and apparatus may be employed without departing from the true scope of the invention defined in the following claims.

What is claimed is:

1. A method of controlling efficacy of a food processing system, the method comprising:
responsive to receiving set points from an automatic expert system, operating an adaptive controller to perform steps comprising:
comparing the set points from the automatic expert system to process variables for the food processing system;
generating an error signal indicating a difference between the compared process variables and the set points; and
adjusting control settings for the food processing system using the error signal and one or more control parameters, wherein the adaptive controller is configured to automatically modify the control parameters in response to a detected change in at least one of the set points and the process variables.

2. The method of claim 1, wherein the one or more control parameters includes a gain, a time constant, and a delay for the adaptive controller.

3. The method of claim 2, wherein the gain for the adaptive controller is calculated as a function of a relationship between one or more manipulated variables and one or more control variables.

4. The method of claim 1, wherein adjusting the control settings for the food processing system comprises performing at least three step changes to the control settings.

5. The method of claim 4, wherein the at least three step changes correspond to an upper operating range, middle operating range, and lower operating range for the control settings for the food processing system.

6. The method of claim 1, wherein the process variables include a pressure variable, temperature variable, solubility variable, fluid density variable, and residence time variable for the food processing system.

7. The method of claim 1, wherein the control settings modify at least one of a food flow rate, a reagent flow rate, and a heating fluid flow rate.

8. The method of claim 1, wherein the automatic expert system is configured to provide subsequent set points to the adaptive controller only after the process variables for the food processing system have reached a steady state and quality variables become available through an instrumented system downstream of the food processing system.

9. An adaptive controller configured to control efficacy of a food processing system, the controller comprising:
a computer readable storage medium including a plurality of instructions;
a processor, which, when executing the plurality of instructions, is configured to:
receive set points from an automatic expert system;
compare the set points from the automatic expert system to process variables for the food processing system, wherein comparing comprises generating an error signal indicating a difference between the process variables and the set points; and
adjust control settings for the food processing system using the error signal and one or more control parameters, wherein the adaptive controller is configured to automatically modify the control parameters in response to a detected change in at least one of the set points and the process variables.

10. The adaptive controller of claim 9, wherein the one or more control parameters includes a gain, a time constant, and a delay for the adaptive controller.

11. The adaptive controller of claim 10, wherein the gain for the adaptive controller is calculated as a function of a relationship between one or more manipulated variables and one or more control variables.

12. The adaptive controller of claim 9, wherein adjusting the control settings for the food processing system comprises performing at least three step changes to the control settings.

13. The adaptive controller of claim 12, wherein the at least three step changes correspond to an upper operating range, middle operating range, and lower operating range for the control settings for the food processing system.

14. The adaptive controller of claim 9, wherein the process variables include a pressure variable, temperature variable, solubility variable, fluid density variable, and residence time variable for the food processing system.

15. The adaptive controller of claim 9, wherein the control settings modify at least one of a food flow rate, a reagent flow rate, and a heating fluid flow rate.

16. The adaptive controller of claim 9, wherein the automatic expert system is configured to provide subsequent set points to the adaptive controller only after the process variables for the food processing system have reached a steady state and quality variables become available through an instrumented system downstream of the food processing system.

17. A method for generating set points used by an adaptive controller for controlling efficacy of a food processing system, the method comprising:
receiving process variables from the food processing system;
comparing the process variables from the food processing system to quality control metrics derived from the food processing system;
based upon a comparison of the process variables and the quality control metrics, generating the set points used by the adaptive controller for controlling the food processing system; and
providing the set points to the adaptive controller.

18. The method of claim 17, wherein the process variables include a pressure variable, temperature variable, solubility variable, fluid density variable, and residence time variable for the food processing system.

19. The method of claim 17, wherein the quality control metrics include at least one of a pH metric and microbiological data provided by an instrumented system downstream of the food processing system.

20. The method of claim 19, wherein the microbiological data includes a bacteria kills metric.

21. The method of claim 17, further comprising:
providing subsequent set points to the adaptive controller only after the process variables for the food processing system have reached a steady state and quality variables become available through an instrumented system downstream of the food processing system.

22. The method of claim 17, wherein comparing the process variables to quality control metrics and generating the set points comprises:
providing the process variables and quality control metrics to a neural network configured to perform the comparison of the process variables and quality control metrics and generate the set points based upon the comparison.

23. The method of claim 22, wherein the neural network includes a plurality of perceptrons arranged as a multilayer network of perceptrons, wherein back-propagation of signals in the multilayer network of perceptrons is used to perform the comparison of the process variables and quality control metrics and generation of the set points based upon the comparison, wherein the quality control metrics includes microbiological data.

24. The method of claim 23, wherein the microbiological data includes bacteria kills.

25. An automatic expert system configured to generate set points used by an adaptive controller for controlling efficacy of a food processing system, comprising:
a computer readable storage medium including a plurality of instructions;
a processor, which, when executing the plurality of instructions, is configured to:
receive process variables from the food processing system;
compare the process variables from the food processing system to quality control metrics derived from the food processing system;
based upon a comparison of the process variables and the quality control metrics, generate the set points used by the adaptive controller for controlling the food processing system; and
provide the set points to the adaptive controller.

26. The automatic expert system of claim 25, wherein the process variables include a pressure variable, temperature variable, solubility variable, fluid density variable, and residence time variable for the food processing system.

27. The automatic expert system of claim 25, wherein the quality control metrics include at least one of a pH metric, and microbiological data provided by an instrumented system downstream of the food processing system.

28. The automatic expert system of claim 27, wherein the microbiological data includes a bacteria kills metric.

29. The automatic expert system of claim 25, wherein the processor is further configured to:
provide subsequent set points to the adaptive controller only after the process variables for the food processing system have reached a steady state and quality variables become available through an instrumented system downstream of the food processing system.

30. The automatic expert system of claim 25, wherein the processor comprises a neural network which is configured to receive the process variables and quality control metrics, wherein the neural network performs the comparison of the process variables and quality control metrics and generates the set points based upon the comparison.

31. The automatic expert system of claim 30, wherein the neural network includes a plurality of perceptrons arranged as a multilayer network of perceptrons, wherein back-propagation of signals in the multilayer network of perceptrons is used to perform the comparison of the process variables and quality control metrics and generation of the set points based upon the comparison.

32. A food processing system, comprising:
a chamber in which food processing is performed;
a plurality of sensors configured to provide process variables for the food processing system;
an automatic expert system configured to:
receive the process variables for the food processing system;
receive quality control metric for the food processing system;
compare the process variables from the food processing system to quality control metrics derived from the food processing system; and
based upon a comparison of the process variables and the quality control metrics, generate the set points used by the adaptive controller for controlling the food processing system; and
an adaptive controller configured to:
receive the set points from the automatic expert system;
compare the set points to the process variables for the food processing system, wherein comparing comprises generating an error signal indicating a difference between the process variables and the set points; and
adjust control settings for the food processing system using the error signal and one or more control parameters, wherein the adaptive controller is configured to automatically modify the control parameters in response to a detected change in at least one of the set points and the process variables.

33. The food processing system of claim 32, wherein the one or more control parameters includes a gain, a time constant, and a delay for the adaptive controller.

34. The food processing system of claim 33, wherein the gain for the adaptive controller is calculated as a function of a relationship between one or more manipulated variables and one or more control variables.

35. The food processing system of claim 32, wherein the process variables include a pressure variable, temperature variable, solubility variable, fluid density variable, and residence time variable for the food processing system.

36. The food processing system of claim 32, wherein the control settings modify at least one of a food flow rate, a reagent flow rate, and a heating fluid flow rate for the chamber in which the food processing system is performed.

37. The food processing system of claim 32, wherein the automatic expert system is configured to provide subsequent set points to the adaptive controller only after the process variables for the food processing system have reached a steady state and quality variables become available through an instrumented system downstream of the food processing system.

38. The food processing system of claim 32, wherein the quality control metrics include at least one of a pH metric and microbiological data provided by an instrumented system downstream of the food processing system.

39. The food processing system of claim 38, wherein the microbiological data includes a bacteria kills metric.

40. The food processing system of claim 32, wherein the automatic expert system comprises a neural network which is configured to receive the process variables and quality control metrics, wherein the neural network performs the comparison of the process variables and quality control metrics and generates the set points based upon the comparison.

41. The food processing system of claim 40, wherein the neural network includes a plurality of perceptrons arranged as a multilayer network of perceptrons, wherein back-propagation of signals in the multilayer network of perceptrons is used to perform the comparison of the process variables and quality control metrics and generation of the set points based upon the comparison.
